(19) United States
(12) Patent Application Publication
(10) Pub. No.: US 2010/0317672 A1
(43) Pub. Date: Dec. 16, 2010

(54) Monomaleate Monohydrate Salt of a 5HT1A Receptor Antagonist

(75) Inventors: Clare Louise Anderton, Hertfordshire (GB); Grahame Robert Woollam, Hertfordshire (GB)

Correspondence Address: GlaxoSmithKline;GLOBAL PATENTS -US, UW2220, P. O. BOX 1539, KING OF PRUSSIA, PA, 19406-0939, US

(73) Assignee: GLAXO GROUP LIMITED, GREENFORD, MIDDLESEX (GB)

(21) Appl. No.: 12/812,973

(22) PCT Filed: Jan. 15, 2009
(86) PCT No.: PCT/EP2009/050401

§ 371 Date: Jul. 15, 2010

(30) Foreign Application Priority Data
Jan. 17, 2008 (GB) 0800840.1

(51) Int. Cl. A61K 31/496; C07D 471/04; A61P 15/12; A61P 15/00; A61P 25/24; A61P 25/18

(52) U.S. Cl. 514/253.03; 544/363

(57) ABSTRACT

The present invention relates to a monomaleate monohydrate salt of 7-methyl-6-{2-[4-(2-methyl-5-quinolinyl)-1-piperazinyl]ethyl}-4,5-dihydroimidazo[1,5-a]quinoline-3-carboxamide, uses of the salt as a medicament in the treatment of sexual dysfunction and pharmaceutical compositions and dosage forms comprising the salt.

The present invention relates to a monomaleate monohydrate salt of 7-methyl-6-{2-[4-(2-methyl-5-quinolinyl)-1-piperazinyl]ethyl}-4,5-dihydroimidazo[1,5-a]quinoline-3-carboxamide, uses of the salt as a medicament in the treatment of sexual dysfunction and pharmaceutical compositions and dosage forms comprising the salt.

The compound 7-methyl-6-{2-[4-(2-methyl-5-quinolinyl)-1-piperazinyl]ethyl}-4,5-dihydroimidazo[1,5-a]quinoline-3-carboxamide of formula (A) and its dihydrochloride salt are described in International Patent Publication WO2006/024517 (see Example 134 therein) incorporated herein by reference.

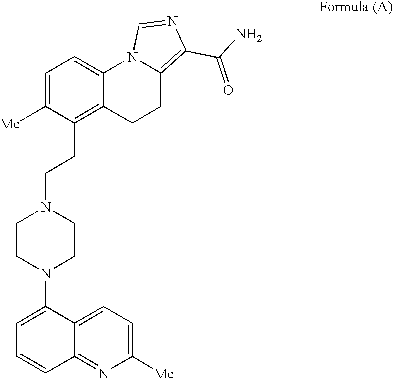

We have identified a novel salt of the compound of formula (A) which is advantageous over the prior art.

Therefore, in a first aspect, the invention provides a monomaleate monohydrate salt of 7-methyl-6-{2-[4-(2-methyl-5-quinolinyl)-1-piperazinyl]ethyl}-4,5-dihydroimidazo[1,5-a]quinoline-3-carboxamide.

Problems associated with the free base and dihydrochloride salt of 7-methyl-6-{2-[4-(2-methyl-5-quinolinyl)-1-piperazinyl]ethyl}-4,5-dihydroimidazo[1,5-a]quinoline-3-carboxamide previously disclosed have been identified. Both have been shown to exist in a number of forms and attempts at reproducibly isolating a single discrete form on scale-up have been unsuccessful. In addition, the free base is insoluble and is therefore not readily bioavailable and the hygroscopic dihydrochloride salt shows variable stoichiometry, leading to uncertainty in the amount of the drug substance when formulating a tablet and giving rise ultimately to a possible risk to the patient.

The monomaleate monohydrate salt of 7-methyl-6-{2-[4-(2-methyl-5-quinolinyl)-1-piperazinyl]ethyl}-4,5-dihydroimidazo[1,5-a]quinoline-3-carboxamide is therefore particularly advantageous over both the free base and the dihydrochloride salt because it can be made and isolated on a large scale as a single discrete form.

The monomaleate monohydrate salt may be amorphous or crystalline. In an embodiment, the monomaleate monohydrate salt is crystalline.

In an embodiment, the monomaleate monohydrate salt has a crystal structure substantially characterised by the following XRPD peak list:

| Position | |
|---|---|
| 2θ/° | |
| (±0.2° 2θ) | d-spacing/Å |
| 5.2 | 17.1 |
| 9.4 | 9.4 |
| 10.3 | 8.6 |
| 10.9 | 8.1 |
| 12.8 | 6.9 |
| 13.1 | 6.7 |
| 13.3 | 6.7 |
| 14.7 | 6.0 |
| 15.4 | 5.7 |
| 16.3 | 5.5 |
| 17.1 | 5.2 |
| 20.6 | 4.3 |
| 21.3 | 4.2 |
| 21.9 | 4.1 |
| 22.2 | 4.0 |
| 25.9 | 3.4 |
| 26.5 | 3.4 |
| 26.7 | 3.3 |
| 31.1 | 2.9 |

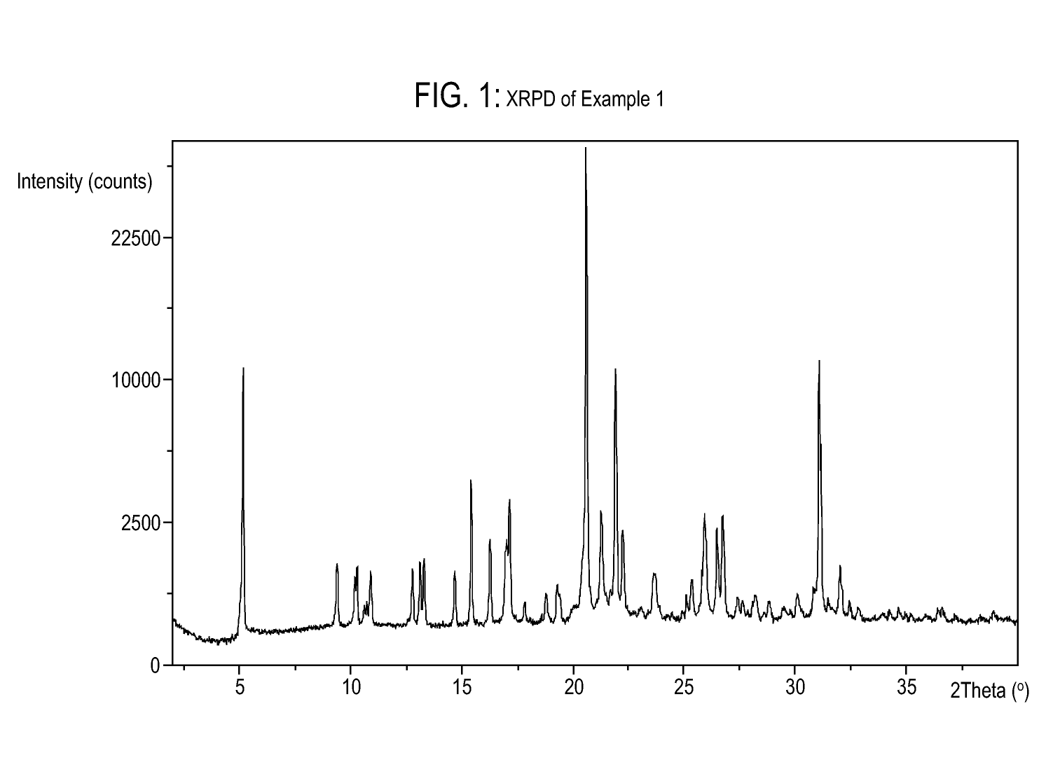

In a further embodiment, the monomaleate monohydrate salt has a crystal structure characterised in that it has an XRPD pattern substantially the same as in FIG. 1.

The X-ray powder diffraction (XRPD) data were acquired on a PANalytical X′Pert Pro powder diffractometer, model PW3040/60 using an XCelerator detector. The acquisition conditions were: radiation: Cu Kα, generator tension: 40 kV, generator current: 45 mA, start angle: 2.0° 2θ, end angle: 40.0° 2θ, step size: 0.0167° 2θ, time per step: 31.75 seconds. The sample was prepared by mounting a few milligrams of sample on a silicone wafer (zero background) plate, resulting in a thin layer of powder. Peak positions were measured using Highscore software.

In an embodiment, the monomaleate monohydrate salt has a crystal structure characterised by a dehydration endotherm with an onset of 45° C. followed by melting of the dehydrated monomaleate monohydrate with an onset of 222° C. in the DSC thermogram.

The DSC thermogram of the monomaleate monohydrate salt was obtained using a TA Q1000 calorimeter. The sample was weighed into an aluminium pan, a pan lid placed on top and lightly crimped without sealing the pan. The experiment was conducted using a heating rate of 10° C. min⁻¹.

In an embodiment, the monomaleate monohydrate salt has a crystal structure characterised by the following absorption peaks in the infrared spectrum of the solid product at about 3573, 3460, 3298, 1662, 1579, 1494, 1442, 1088, 973, 866, 810, and 748 cm⁻¹.

Slight variations in observed peaks are expected based on the specific spectrometer employed and the analyst's sample preparation technique. Some margin of error is present in each of the peak assignments reported above.

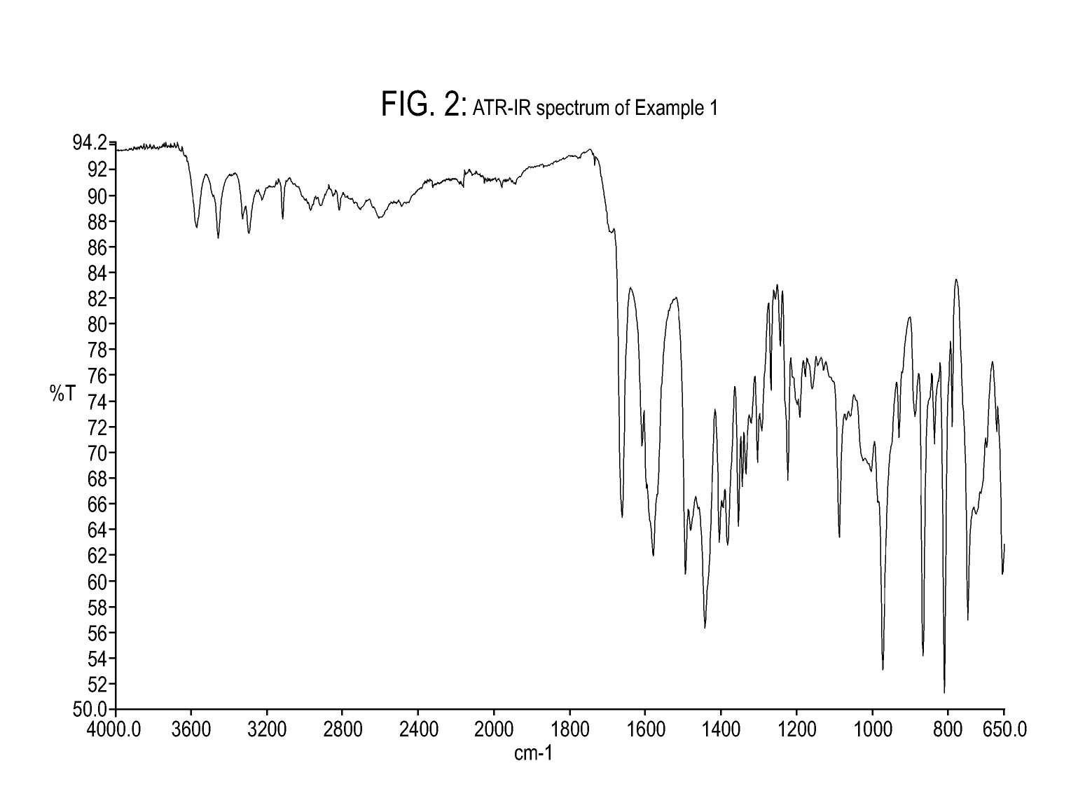

In a further embodiment, the monomaleate monohydrate salt has a crystal structure characterised in that it has an infra red spectrum substantially the same as in FIG. 2.

The infra red spectrum was acquired after 16 scans at 2 cm⁻¹ resolution on a PerkinElmer Spectrum One FTIR using a Universal (single bounce diamond) ATR sampling accessory. The monomaleate monohydrate salt as defined in the first aspect and embodiments thereof, is hereinafter referred to as the “salt of the invention”.

The salt of the invention includes all suitable isotopic variations thereof. An isotopic variation is defined as one in which at least one atom is replaced by an atom having the same atomic number but an atomic mass different from the atomic mass usually found in nature. Examples of isotopes that can be incorporated include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations, for example, those in which a radioactive isotope such as ³H or ¹⁴C is incorporated, are useful in drug and/or substrate tissue distribution studies. Tritiated, i.e. ³H, and carbon-14, i.e. ¹⁴C, isotopes are suitable for their ease of preparation and detectability. Further, substitution with isotopes such as deuterium, i.e. ²H, may afford certain therapeutic advantages resulting from greater metabolic stability, for example, increased in vivo half-life or reduced dosage requirements and hence may be suitable in some circumstances. Isotopic variations can generally be prepared by conventional procedures such as by the illustrative methods or by the preparations described in the Examples and Descriptions hereafter using appropriate isotopic variations of suitable reagents.

The salt of the invention is an effective antagonist of the 5-HT_{1A} receptor. In addition, the salt of the invention has high selectivity for the 5-HT_{1A} receptor over the 5-HT_{1B} receptor, i.e. the salt is a better antagonist of the 5-HT_{1A} receptor than it is antagonist of the 5-HT_{1B} receptor.

It is believed that the salt of the invention may be useful for the treatment of diseases and conditions mediated by activation of the 5-HT_{1A} receptor.

Therefore, according to an embodiment, the invention provides the salt of the invention for use as a medicament, preferably a human medicament.

According to a further aspect, the invention provides a method of treatment or prevention of diseases and conditions mediated by activation of the 5-HT_{1A} receptor in humans, which comprises administering to the sufferer a therapeutically safe and effective amount of a compound of formula (A).

According to a further aspect, the invention provides the use of the salt of the invention in the manufacture of a medicament for treating or preventing a disease or condition mediated by activation of the 5-HT_{1A} receptor.

In an embodiment, diseases or conditions that may be mediated by activation of the 5-HT_{1A} receptor are selected from the list consisting of: [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Psychotic disorders for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9).
ii) Depression and mood disorders for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90).
iii) Anxiety disorders for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00).
iv) Substance-related disorders for example Substance Use Disorders (including Substance Dependence, Substance Craving and Substance Abuse); Substance-Induced Disorders (including Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders (including Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9)); Amphetamine (or Amphetamine-Like)-Related Disorders (for example Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9)); Caffeine Related Disorders (including Caffeine Intoxication (305.90), Caffeine-Induced Anxiety Disorder, Caffeine-Induced Sleep Disorder and Caffeine-Related Disorder Not Otherwise Specified (292.9)); Cannabis-Related Disorders (including Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9)); Cocaine-Related Disorders (including Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9)); Hallucinogen-Related Disorders (including Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9)); Inhalant-Related Disorders (including Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9)); Nicotine-Related Disorders (including Nicotine Dependence (305.1), Nicotine Withdrawal (292.0) and Nicotine-Related Disorder Not Otherwise Specified (292.9)); Opioid-Related Disorders (including Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9)); Phencyclidine (or Phencyclidine-Like)-Related Disorders (including Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9)); Sedative-, Hypnotic-, or Anxiolytic-Related Disorders (including Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9)); Polysubstance-Related Disorder (including Polysubstance Dependence (304.80)); and Other (or Unknown) Substance-Related Disorders (including Anabolic Steroids, Nitrate Inhalants and Nitrous Oxide).
v) Sleep disorders for example primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type). vi) Eating disorders such as Anorexia Nervosa (307.1) (including the subtypes Restricting Type and Binge-Eating/Purging Type); Bulimia Nervosa (307.51) (including the subtypes Purging Type and Nonpurging Type); Obesity; Compulsive Eating Disorder; Binge Eating Disorder; and Eating Disorder Not Otherwise Specified (307.50).
vii) Autism Spectrum Disorders including Autistic Disorder (299.00), Asperger's Disorder, Rett's Disorder, Childhood Disintegrative Disorder and Pervasive Developmental Disorder Not Otherwise Specified.
viii) Attention-Deficit/Hyperactivity Disorder (including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9)); Hyperkinetic Disorder; Disruptive Behaviour Disorders such as Conduct Disorder (including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).
ix) Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).
x) Enhancement of cognition including the treatment of cognition impairment in other diseases such as schizophrenia, bipolar disorder, depression, other psychiatric disorders and psychotic conditions associated with cognitive impairment, e.g. Alzheimer's disease.
xi) Sexual dysfunctions such as Sexual Desire Disorders (including Hypoactive Sexual Desire Disorder (302.71) and Sexual Aversion Disorder (302.79)); sexual arousal disorders (including Female Sexual Arousal Disorder (302.72) and Male Erectile Disorder (302.72)); orgasmic disorders (including Female Orgasmic Disorder (302.73), Male Orgasmic Disorder (302.74) and Premature Ejaculation (302.75)); sexual pain disorder (including Dyspareunia (302.76) and Vaginismus (306.51)); Sexual Dysfunction Not Otherwise Specified (302.70); paraphilias (including Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9)); gender identity disorders (including Gender Identity Disorder in Children (302.6) and Gender Identity Disorder in Adolescents or Adults (302.85)); and Sexual Disorder Not Otherwise Specified (302.9).

In a further embodiment, diseases or conditions that may be mediated by activation of the 5-HT_{1A} receptor are selected from the group i) Psychotic disorders, ii) Depression and mood disorders, iii) Anxiety disorders and xi) Sexual dysfunctions.

In a further embodiment, diseases or conditions that may be mediated by activation of the 5-HT_{1A} receptor are selected from the group i) Depression and mood disorders, iii) Anxiety disorders and xi) Sexual dysfunctions.

In a further embodiment, diseases or conditions that may be mediated by activation of the 5-HT_{1A} receptor are sexual dysfunctions.

In a further embodiment, the sexual dysfunction is premature ejaculation.

The salt of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The salt of the invention may be used in combination with antidepressants to treat or prevent depression and mood disorders.

The salt of the invention may be used in combination with the following agents to treat or prevent bipolar disease: i) mood stabilisers; ii) antipsychotics; and iii) antidepressants.

The salt of the invention may be used in combination with the following agents to treat or prevent anxiety disorders: i) anxiolytics; and ii) antidepressants.

The salt of the invention may be used in combination with the following agents to treat or prevent male sexual dysfunction: i) phosphodiesterase V inhibitors, for example vardenafil and sildenafil; ii)) dopamine agonists/dopamine antagonists/dopamine transport inhibitors for example apomorphine and buproprion; iii) alpha adrenoceptor antagonists for example phentolamine; iv) prostaglandin agonists for example alprostadil; v) androgen receptor modulators such as testosterone; vi) serotonin agonists/antagonists/modulators/serotonin transporter inhibitors for example serotonin reuptake inhibitors; vii) noradrenaline transport inhibitors for example reboxetine; viii) oxytocin receptor antagonists; (ix) sodium and calcium channel inhibitors/blockers; and (x) opioid receptor antagonists.

The salt of the invention may be used in combination with the same agents specified for male sexual dysfunction to treat or prevent female sexual dysfunction, and in addition an estrogen agonist such as estradiol.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine, sertraline femoxetine, fluvoxamine, indalpine and zimeldine); dual serotonin/noradrenaline reuptake inhibitors (such as venlafaxine, duloxetine and milnacipran); Noradrenaline reuptake inhibitors (such as reboxetine and venlafaxine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

In addition the salt of the invention may be administered in combination with 5-HT₃ antagonists (such as ondansetron, granisetron and metoclopramide); serotonin agonists (such as sumatriptan, rauwolscine, yohimbine and metoclopramide); and NK-1 antagonists.

It will be appreciated that the above mentioned combinations may be administered simultaneously (either in the same or different pharmaceutical formulations), separately or sequentially.

It will be appreciated that references herein to “treatment” extend to prophylaxis, prevention of recurrence and suppression or amelioration of symptoms (whether mild, moderate or severe) as well as the treatment of established conditions.

The salt of the invention will normally, but not necessarily, be formulated into pharmaceutical compositions prior to administration to a patient by an appropriate route. Accordingly, in another aspect, the invention provides pharmaceutical compositions comprising a salt of the invention and one or more pharmaceutically-acceptable excipients.

As used herein, “pharmaceutically-acceptable excipient” means any pharmaceutically acceptable material present in the pharmaceutical composition or dosage form other than the salt of the invention. Typically the material gives form, consistency and performance to the pharmaceutical composition.

The salt of the invention may be administered alone, but it will generally be administered as a formulation in conjunction with one or more pharmaceutically acceptable excipients, selected with regard to the intended route of administration and standard pharmaceutical practice. Therefore according to a further aspect, the invention provides a pharmaceutical composition comprising a salt of the invention, in association with one or more pharmaceutically acceptable excipients(s), diluents(s) and/or excipient(s). The excipient must be “acceptable” in the sense of being compatible with the other ingredients of the composition and not deleterious to the recipient thereof.

Such pharmaceutical compositions of the invention may be prepared and packaged in bulk form wherein a safe and effective amount of the salt of the invention can be dispensed and then given to the patient such as with powders or syrups. Alternatively, the pharmaceutical compositions of the invention may be prepared and packaged as oral dosage forms wherein each physically discrete dosage form contains a safe and effective amount of the salt of the invention. Accordingly, in another aspect, the invention provides oral dosage forms comprising pharmaceutical compositions of the invention. Each discrete dosage form contains the salt of the invention comprising from 0.5 mg to 25 mg of the free base. In another aspect, each discrete dosage form contains the salt of the invention comprising from 1 mg to 25 mg of the free base. In another aspect, each discrete dosage form contains the salt of the invention comprising from 1 mg to 20 mg of the free base. In another aspect, each discrete dosage form contains the salt of the invention comprising from 1 mg to 15 mg of the free base. In another aspect, each discrete dosage form contains the salt of the invention comprising from 1 mg to 5 mg of the free base.

It will be recognised by one of skill in the art that the optimal quantity and spacing of individual dosages of the salt of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e. the number of doses of salt of the invention given per day for a defined number of days, can be ascertained by those skilled in the art using conventional course of treatment determination tests.

The compositions of the invention will typically be formulated into dosage forms which are adapted for administration to the patient by the desired route of administration. For example, dosage forms include those adapted for (1) oral administration such as tablets, capsules, caplets, pills, lozenges, powders, syrups, elixirs, suspensions, solutions, emulsions, sachets and cachets; (2) parenteral administration such as sterile solutions, suspensions, implants and powders for reconstitution; (3) transdermal administration such as transdermal patches; (4) rectal and vaginal administration such as suppositories, pessaries and foams; (5) inhalation and intranasal such as dry powders, aerosols, suspensions and solutions (sprays and drops); (6) topical administration such as creams, ointments, lotions, solutions, pastes, drops, sprays, foams and gels; (7) ocular administration such as drops, ointment, sprays, suspensions and inserts; (8) buccal and sublingual administration such as lozenges, patches, sprays, drops, chewing gums and tablets.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the salt of the invention once administered to the patient from one organ, or portion of the body, to another organ, or portion of the body. Certain pharmaceutically-acceptable excipients may be chosen for their ability to enhance patient compliance. Certain pharmaceutically-acceptable excipients may be chosen for their ability to facilitate the release of the salt of the invention at the appropriate rate to treat the condition.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavouring agents, flavour masking agents, colouring agents, anticaking agents, humectants, chelating agents, plasticizers, viscosity increasing agents, rate modifying agents, antioxidants, preservatives, stabilizers, surfactants and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to determine suitable pharmaceutically-acceptable excipients in appropriate amounts for use with the salt of the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press). The pharmaceutical compositions of the invention may be prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of the salt of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch and pre-gelatinized starch), gelatin, acacia, sodium alginate, alginic acid, tragacanth, guar gum, povidone, and cellulose and its derivatives (e.g. hydroxypropyl methyl cellulose). The oral solid dosage form may further comprise a disintegrant. Suitable disintegrants include starches, crospovidone, sodium starch glycolate, cros-carmellose, alginic acid, and sodium carboxymethyl cellulose. The oral solid dosage form may further comprise a lubricant. Suitable lubricants include stearic acid, magnesium stearate, calcium stearate, and sodium dodecyl sulphate. The oral solid dosage form may further comprise a glidant such as talc and colloidal silicon dioxide. The oral solid dosage form may further comprise an outer coating which may have cosmetic or functional properties.

It will be appreciated that the invention includes the following further aspects. The diseases and conditions described above extend, where appropriate, to these further aspects.
i) The salt of the invention for use in treating or preventing sexual dysfunction, such as premature ejaculation.
ii) A method of treatment or prevention of sexual dysfunction (such as premature ejaculation) in a human comprising administering an effective amount of the salt of the invention.

The invention is illustrated by the Examples described below.

Compounds are named using ACD/Name PRO6.02 chemical naming software (Advanced Chemistry Development Inc., Toronto, Ontario, M5H2L3, Canada).

### EXAMPLE 1

### Preparation of the Monomaleate Monohydrate Salt of 7-methyl-6-{2-[4-(2-methyl-5-quinolinyl)-1-piperazinyl]ethyl}-4,5-dihydroimidazo[1,5-a]quinoline-3-carboxamide

A stirred slurry of 7-methyl-6-{2-[4-(2-methyl-5-quinolinyl)-1-piperazinyl]ethyl}-4,5-dihydroimidazo[1,5-a]quinoline-3-carboxamide (702 g) in dimethyl sulfoxide (6.51) was heated to 75±5° C. for approximately 10 min before maleic acid (212 g) was added. The mixture was heated at 75±5° C. for approximately 1.5 h during which time the slurry dissolved to give a solution. The solution was then transferred through a 5 μm filter into a second vessel. Dimethyl sulfoxide (0.5 L) was heated to 70±5° C. in the first vessel before being transferred to the second vessel via the previously used 5 μm filter. Water (3.5 L) was heated to 70±5° C. in the first vessel before being transferred to the second vessel via the previously used 5 μm filter. The contents of the second vessel were adjusted to 67±3° C. and seeded (2 g). For a preparation of seed material see Example 2. The contents of the vessel were cooled to 22±3° C. over approximately 45 min and filtered. The filter cake was washed with acetone (3×2.8 L), briefly pulled dry and then dried in vacuo at 50±5° C. for approximately 15 h. Upon removal from the oven the product was stood at ambient temperature for >3 h to constant weight (568 g).

The percentage of water, by weight, of the product of Example 1 was determined using coulometric Karl Fisher titration using a Metrohm titrator fitted with headspace autosampler. The amount of water was determined to be 2.9% w/w. This corresponds to the expected stoichiometric amount of 2.9% w/w.

The percentage of maleate counter ion, by weight, of the product of Example 1 was determined using ion chromatography using a Dionex ICS-2000 and a Dionex IPAC column. The amount of maleate ion was determined to be 18.5% w/w. This corresponds well to the expected stoichiometric amount of 18.9% w/w.

The product of Example 1 was characterised as follows:

### X-Ray Powder Diffraction (XRPD)

The X-ray powder diffraction (XRPD) data were acquired on a PANalytical X′Pert Pro powder diffractometer, model PW3040/60 using an XCelerator detector. The acquisition conditions were: radiation: Cu Kα, generator tension: 40 kV, generator current: 45 mA, start angle: 2.0° 2θ, end angle: 40.0° 2θ, step size: 0.0167° 2θ, time per step: 31.75 seconds. The sample was prepared by mounting a few milligrams of sample on a Si wafer (zero background) plate, resulting in a thin layer of powder. The XRPD pattern is shown in FIG. 1. Characteristic XRPD angles and d-spacings are recorded in the table below. The margin of error is approximately ±0.1° 2θ for each of the peak assignments. Peak positions were measured using Highscore software.

| Position | |
|---|---|
| 2θ/° | |
| (±0.1° 2θ) | d-spacing/Å |
| 5.2 | 17.1 |
| 9.4 | 9.4 |
| 10.3 | 8.6 |
| 10.9 | 8.1 |
| 12.8 | 6.9 |
| 13.1 | 6.7 |
| 13.3 | 6.7 |
| 14.7 | 6.0 |
| 15.4 | 5.7 |
| 16.3 | 5.5 |
| 17.1 | 5.2 |
| 20.6 | 4.3 |
| 21.3 | 4.2 |
| 21.9 | 4.1 |
| 22.2 | 4.0 |
| 25.9 | 3.4 |
| 26.5 | 3.4 |
| 26.7 | 3.3 |
| 31.1 | 2.9 |

### ATR-IR

The infra red spectrum was acquired after 16 scans at 2 cm⁻¹ resolution on a PerkinElmer Spectrum One FTIR using a Universal (single bounce diamond) ATR-IR sampling accessory. The ATR-IR spectrum is shown in FIG. 2.

Characteristic absorption peaks are observed at about: 3573, 3460, 3298, 1662, 1550-1600, 1579, 1494, 1442, 1088, 973, 866, 810, and 748 cm⁻¹.

### Thermal Analysis

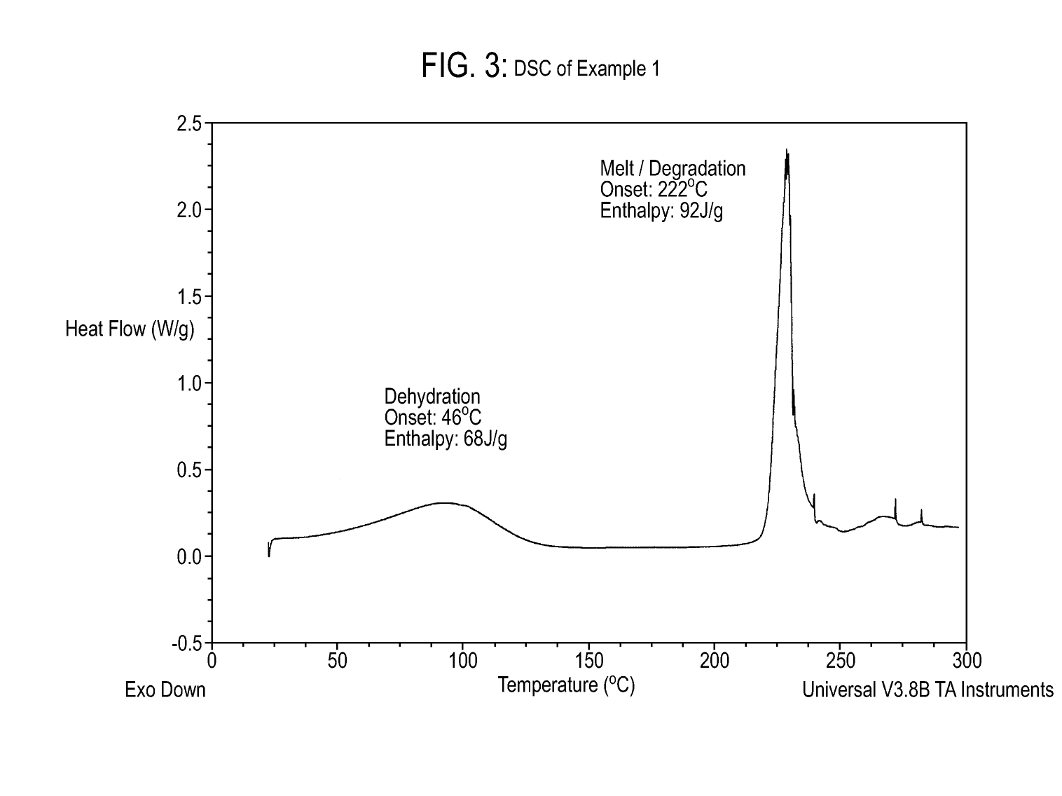

The DSC thermogram of the product was obtained using a TA Q1000 calorimeter. The sample was weighed into an aluminium pan, a pan lid placed on top and lightly crimped without sealing the pan. The experiment was conducted using a heating rate of 10° C. min⁻¹. The DSC thermogram of the product from Example 1 is shown in FIG. 3.

Dehydration onset occurred 46° C. Melt/degradation onset occurred at 222° C.

### EXAMPLE 2

### A Preparation of 7-methyl-6-{2-[4-(2-methyl-5-quinolinyl)-1-piperazinyl]ethyl}-4,5-dihydroimidazo[1,5-a]quinoline-3-carboxamide monomaleate monohydrate

A first sample was prepared as follows. A stirred slurry of 7-methyl-6-{2-[4-(2-methyl-5-quinolinyl)-1-piperazinyl]ethyl}-4,5-dihydroimidazo[1,5-a]quinoline-3-carboxamide (1.68 g) and maleic acid (0.71 g) in 5% v/v aqueous isopropanol (224 ml) was heated to approximately 70° C. After approximately 75 min, the resultant thin slurry was allowed to cool to approximately 40° C. The temperature cycle (heating to approximately 70° C. and cooling to approximately 40° C.) was repeated four times before the slurry was allowed to cool to room temperature and stirred overnight. The product was filtered and washed with 5% v/v aqueous isopropanol (2×5 ml) and then dried in vacuo at 50° C. to give a product (1.77 g). ¹H NMR analysis of the product showed slightly less than 1 equivalent of maleic acid.

A second sample was prepared as follows. A stirred slurry of 7-methyl-6-{2-[4-(2-methyl-5-quinolinyl)-1-piperazinyl]ethyl}-4,5-dihydroimidazo[1,5-a]quinoline-3-carboxamide (1.45 g) and maleic acid (0.61 g) in 5% v/v aqueous isopropanol (193 ml) was heated to approximately 70° C. After approximately 75 min the resultant slurry was allowed to cool to room temperature and stirred overnight. The product was filtered and washed with 5% v/v aqueous isopropanol (2×3 ml) and then dried in vacuo at 50° C. to give a product (1.58 g). ¹H NMR analysis of the product showed slightly less than 1 equivalent of maleic acid. A third sample was prepared as follows. A stirred slurry of 7-methyl-6-{2-[4-(2-methyl-5-quinolinyl)-1-piperazinyl]ethyl}-4,5-dihydroimidazo[1,5-a]quinoline-3-carboxamide (5.14 g) and maleic acid (2.17 g) in 5% v/v aqueous isopropanol (684 ml) was heated to approximately 70° C. and stirred overnight. The resultant slurry was allowed to cool to room temperature. The product was filtered and washed with 5%/v/v aqueous isopropanol (2×10 ml) and then dried in vacuo at 50° C. to give a product (5.36 g). ¹H NMR of the product showed approximately 0.85 mol maleic acid.

The three samples prepared as indicated above were combined (8.31 g) and warmed in dimethylsulfoxide (70 ml) to approximately 70° C. to give a hazy solution. Maleic acid (167 mg) was added. After a few minutes, water (35 ml) was added and the mixture allowed to cool to room temperature overnight. The product was filtered and washed with 1:1 dimethylsulfoxide/water (16 ml) and then dried in vacuo at 50° C. The product (6.75 g) was suspended in dimethylsulfoxide (57 ml) and heated to approximately 70° C. to give a hazy solution. Maleic acid (433 mg) was added followed by water (28.5 ml) and the mixture warmed to approximately 73° C. After cooling to 40° C. over 90 min the slurry was allowed to cool to room temperature and was stirred overnight. The product was filtered and washed with 5% v/v aqueous isopropanol (2×6.7 ml) and then dried in vacuo at 50° C. to give 7-methyl-6-{2-[4-(2-methyl-5-quinolinyl)-1-piperazinyl]ethyl}-4,5-dihydroimidazo[1,5-a]quinoline-3-carboxamide monomaleate monohydrate (4.71 g).

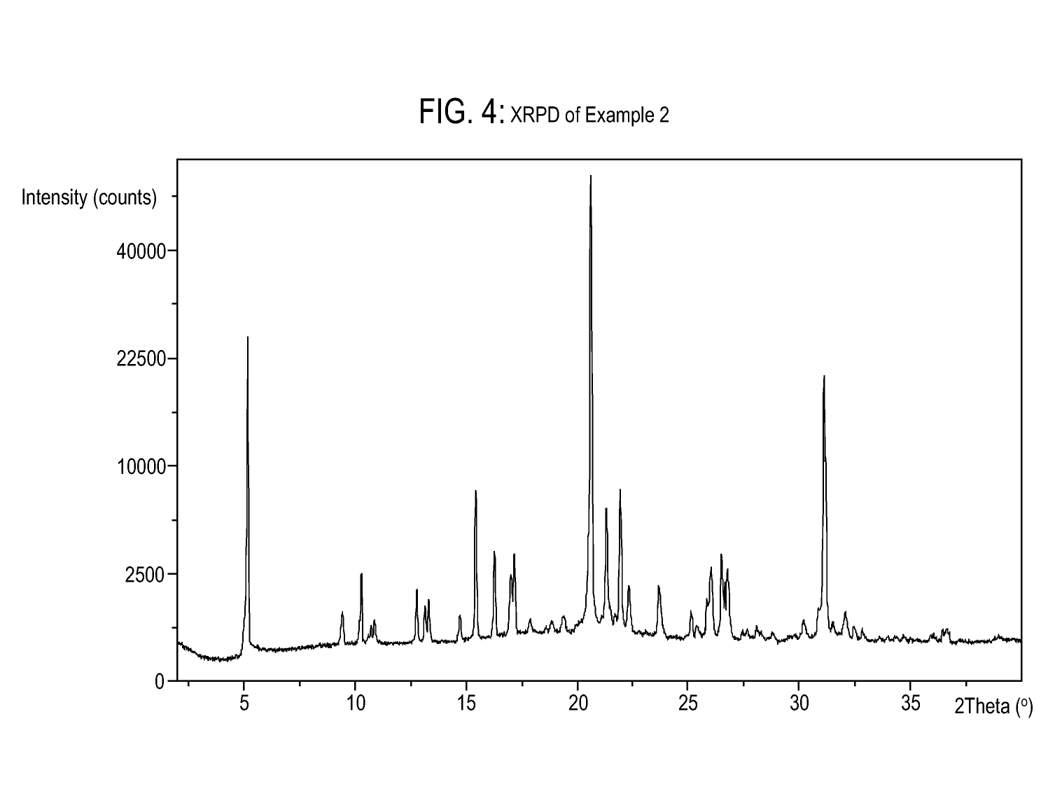

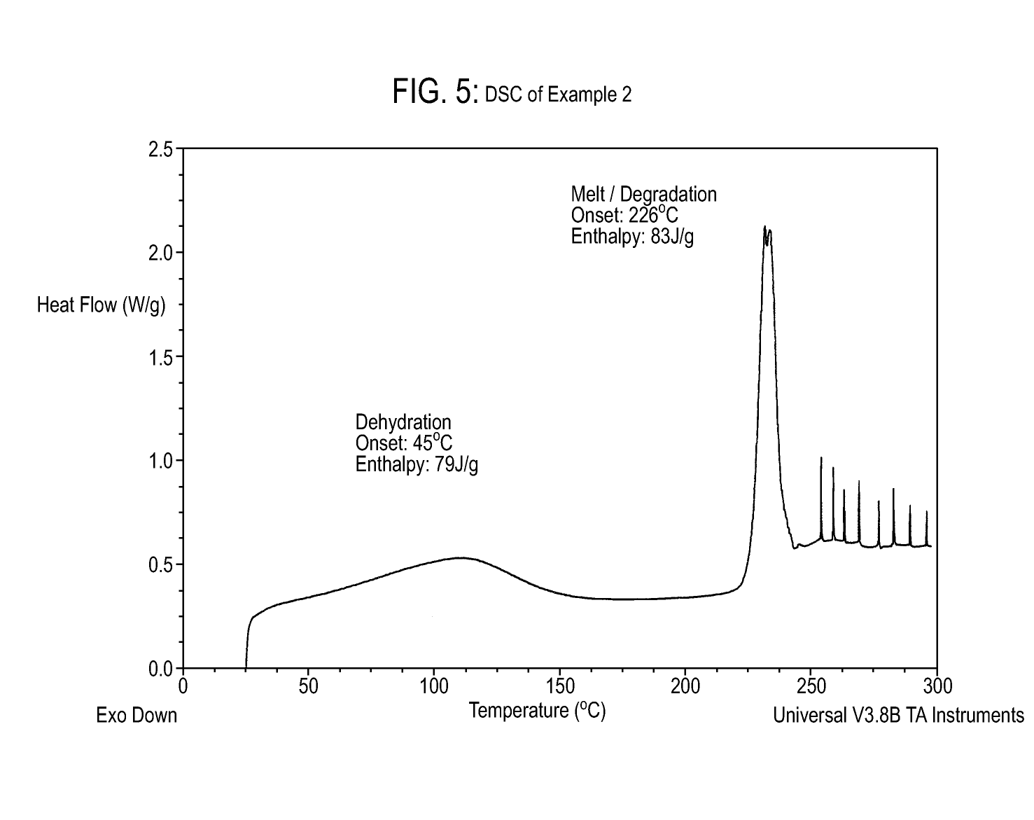

The resulting final product was characterized by XRPD and DSC under the same conditions described for Example 1. (See FIGS. 4 and 5 respectively.)

### EXAMPLE 3

### Preparation of an Immediate Release Aqueous Film Coated Tablet Containing the Salt of the Invention (7-methyl-6-{2-[4-(2-methyl-5-quinolinyl)-1-piperazinyl]ethyl}-4,5-dihydroimidazo[1,5-a]quinoline-3-carboxamide monomaleate monohydrate)

Such a tablet maybe prepared according to procedures known to the skilled person. A particular tablet was prepared as follows:

1. Mix 7-methyl-6-{2-[4-(2-methyl-5-quinolinyl)-1-piperazinyl]ethyl}-4,5-dihydroimidazo[1,5-a]quinoline-3-carboxamide monomaleate monohydrate (salt of the invention) with intragranular excipients HPMC, Mannitol 60, Avicel PH101 and Ac Di Sol® in the amounts shown in Table A.
2. High-shear wet granulate the mixture to obtain granule containing 4% w/w.
3. Dry the wet granule.
4. Mix the resulting granule with extragranular excipients Mannitol SD200, Avicel PH 102, Ac Di Sol® and magnesium stearate in the amounts shown in Table A.
5. Tablet the mixture.
6. Coat resulting tablet.

TABLE A

| | Batch Compositions | | |
|---|---|---|---|
| | (mg/tablet) | | |
| Component | 1 mg | 5 mg | Function |
| 4% w/w Granule | | | |
| Micronised 7-methyl-6-{2-[4-(2- | 1.28¹ | 6.40² | Active |
| methyl-5-quinolinyl)-1- | | | |
| piperazinyl]ethyl}-4,5- | | | |
| dihydroimidazo[1,5-a]quinoline-3- | | | |
| carboxamide monomaleate | | | |
| monohydrate | | | |
| HPMC (Pharmacoat 603) | 1.60 | 7.99 | Binder |
| Mannitol 60 | 14.23 | 71.15 | Filler |
| MCC-Avicel PH101 | 14.39 | 71.95 | Filler |
| Ac Di Sol ® | 0.48 | 2.40 | Disintegrant |
| Extragranular | | | |
| Mannitol SD200 | 199.02 | 71.12 | Filler |
| Microcrystalline cellulose | 105.00 | 105.00 | Filler |
| (Avicel PH102) | | | |
| Ac Di Sol ® | 10.50 | 10.50 | Disintegrant |
| Magnesium stearate | 3.50 | 3.50 | Lubricant |
| Tablet Weight | 350 mg | 350 mg | |
| Opadry Film Coat, Yellow 03B22133 | 10.50 | 10.50 | Film Coating |

¹corresponding to 1.00 mg as 7-methyl-6-{2-[4-(2-methyl-5-quinolinyl)-1-piperazinyl]ethyl}-4,5-dihydroimidazo[1,5-a]quinoline-3-carboxamide
²corresponding to 5.00 mg as 7-methyl-6-{2-[4-(2-methyl-5-quinolinyl)-1-piperazinyl]ethyl}-4,5-dihydroimidazo[1,5-a]quinoline-3-carboxamide

1. A monomaleate monohydrate salt of 7-methyl-6-{2-[4-(2-methyl-5-quinolinyl)-1-piperazinyl]ethyl}-4,5-dihydroimidazo[1,5-a]quinoline-3-carboxamide.

2. The salt according to claim 1 having a crystal structure characterised by the following XRPD peak list:

| Position | |
|---|---|
| 2θ/° | |
| (±0.2° 2θ) | d-spacing/Å |
| 5.2 | 17.1 |
| 9.4 | 9.4 |
| 10.3 | 8.6 |
| 10.9 | 8.1 |
| 12.8 | 6.9 |
| 13.1 | 6.7 |
| 13.3 | 6.7 |
| 14.7 | 6.0 |
| 15.4 | 5.7 |
| 16.3 | 5.5 |
| 17.1 | 5.2 |
| 20.6 | 4.3 |
| 21.3 | 4.2 |
| 21.9 | 4.1 |
| 22.2 | 4.0 |
| 25.9 | 3.4 |
| 26.5 | 3.4 |
| 26.7 | 3.3 |
| 31.1 | 2.9 |

3. The salt according to claim 1 having a crystal structure providing an XRPD pattern substantially the same as in FIG. 1.

4. The salt according to claim 1 having a crystal structure characterised by absorption peaks in an infrared spectrum at about 3573, 3460, 3298, 1662, 1579, 1494, 1442, 1088, 973, 866, 810, and 748 cm⁻¹.

5. The salt according to claim 1 having a crystal structure providing an infra red spectrum substantially the same as in FIG. 2.

6-13. (canceled)

14. A method for the treatment of Psychotic disorders, Depression and mood disorders, Anxiety disorders and Sexual dysfunctions in a human in need thereof comprising administering to said human a therapeutically effective amount of the salt as defined in claim 1.

15. A method for the treatment of Depression and mood disorders, Anxiety disorders and Sexual dysfunctions in a human in need thereof comprising administering to said human a therapeutically effective amount of the salt as defined in claim 1.

16. A method for the treatment of Sexual dysfunctions in a human in need thereof comprising administering to said human a therapeutically effective amount of the salt as defined in claim 1.

17. A method for the treatment of premature ejaculation in a human in need thereof comprising administering to said human a therapeutically effective amount of the salt as defined in claim 1.

18. A pharmaceutical composition comprising the salt as defined in claim 1 in association with one or more pharmaceutically acceptable excipients(s), diluents(s) and/or excipient(s).
